(19) United States
(12) Patent Application Publication
(10) Pub. No.: US 2019/0015032 A1
(43) Pub. Date: Jan. 17, 2019

(54) NEEDLE HOLDERS

(71) Applicant: NSP Tech Pte Ltd, Singapore (SG)

(72) Inventor: Lum Wah LEONG, Singapore (SG)

(21) Appl. No.: 15/718,309

(22) Filed: Sep. 28, 2017

(30) Foreign Application Priority Data
Jul. 14, 2017 (SG) 10201705825R

(51) Int. Cl. A61B 5/15

(52) U.S. Cl. CPC A61B 5/150732

(57) ABSTRACT

When drawing blood, it is useful to always have the needle in a bevel up position but this cannot be guaranteed using conventional syringe barrel/needle assemblies. This problem is solved by providing a needle holder having a proximal end and a distal end, the proximal end being provided with means adapted to secure an integral screw-threaded hub of a needle or a double ended needle, wherein the means comprises a circular wall defining a bore through the proximal end of the needle holder, the wall incorporating a pair of opposed helical ledges, the path of each respective ledge spanning up to half the circumference of the bore, the incline of each ledge being so arranged on the wall as to permit complementary mating contact with a screw-threaded hub of a needle or a double ended needle.

### FIELD OF THE INVENTION

The present invention relates to improvements to needle holders, in particular to needle holders used for drawing blood into ‘blood tubes’ or ‘blood collection tubes’.

### BACKGROUND ART KNOWN TO THE APPLICANT

A hypodermic syringe typically comprises a needle incorporating an integral hub at one end to enable the needle to be connected or mated usually in a ‘luer-lock’ tight manner with the proximal tip or end of a needle holder in the form of a syringe barrel, a syringe barrel and a plunger. Such syringes can be used for intra-muscular injections or to draw material (usually blood) from a patient. Blood collection using hypodermic syringes is well known but today, a needle holder which can be a syringe barrel, vacuum sealed blood collection tube and a double ended needle incorporating an integral hub tends to be used. When the syringe barrel is connected to the double ended needle, the device is known as a pre-attached needle.

When drawing blood, it is possible to draw blood with the needle in any position relative to the skin, however, medical personnel are taught to do so with the bevel of the needle in a position known as ‘bevel up’. Such a position presents the sharpest possible point of the needle for entry into the skin and prevents any possibility of the needle ‘ploughing’ or ‘skimming’ along the skin until the tip of the needle gets a ‘bite’ and goes in. In addition, to minimise the angle of entry of the needle, both opposing finger supports found at the distal end of a syringe barrel ideally need to lie adjacent or close to the skin in a ‘barrel down’ position. With such a ‘bevel up’ and ‘barrel down’ position during the drawing of blood from a patient, pain will tend to be minimised as the needle is inserted into a vein and a blood tube pushed into the syringe barrel.

It is clear therefore, that when a conventional syringe barrel is used, if the connection between the syringe barrel and the needle hub does not always allow for both, a bevel up and a barrel down arrangement during blood collection which can occur with conventional luer-lock screw-threaded arrangements, the patient may suffer unnecessary discomfort.

This poor connection may occur inter alia due to poor machine tolerances that exist between female screw-threads in plastics syringe barrels and male screw-threads in needle hubs.

Ways have been developed to overcome this problem through the use of so-called ‘butterfly needles’ which have a flexible tube that separates the needle from the conventional syringe barrel.

However, such an arrangement presents its own problems as use of such a butterfly needle means that as the blood flows through the flexible tubing, it cools significantly before it is collected in the blood tube and consequently clots faster.

It is therefore an object of the present invention to try and alleviate the aforementioned problem by so arranging matters that a bevel up and a barrel down arrangement is attainable without any unnecessary clotting of the blood.

### STATEMENTS OF THE INVENTION

With the foregoing in view, the invention, although embodied in several different aspects, is so linked as to form part of a single general inventive concept.

Accordingly, the invention in one aspect resides broadly in a needle holder having a proximal end and a distal end, the proximal end being provided with means adapted to secure an integral screw-threaded hub of a needle or a double ended needle, wherein the means comprises a circular wall defining a bore through the proximal end of the needle holder, the wall incorporating a pair of opposed helical ledges, the path of each respective ledge spanning up to half the circumference of the bore, the incline of each ledge being so arranged on the wall as to permit complementary mating contact with a screw-threaded hub of a needle or a double ended needle.

Such an arrangement of the helical ledges (in effect, a pair of female half-threads) will provide a degree of rotational adjustment (through either over or under turn) to an integral screw-threaded hub of a needle or a double ended needle, when fitted to the needle holder especially if the needle holder is a syringe barrel which will enable a bevel up arrangement to always be achievable.

Of course, each ledge could be in the form of one or more castellated projections or one could be a series of castellated projections and the other a continuous ledge however, preferably, each ledge is a continuous ledge.

Preferably, one end of each ledge terminates in a right angled shoulder.

Preferably, one end of each ledge terminates in a bevel. Such an arrangement provides a smooth transition from the wall of the bore to the ledge to guide a male screw-thread of a needle or double ended needle hub.

Preferably, the needle holder has an alignment guide. This has the advantage of enabling a user to align a needle or double ended needle fitted to the needle holder in a bevel up position accurately.

Preferably, each ledge has a substantially uniform cross-section throughout its length. In such form, the cross-section is that of a truncated cone.

Preferably, the needle holder is made of plastics material with a suitable Young's modulus. Such an arrangement will help provide the degree of flexibility needed in the helical ledges to allow the requisite over turn or under turn to enable a bevel up position of a mated needle or double ended needle hub with the needle holder to be achieved. In such form the plastics material is polypropylene.

Preferably, a gap overlies each end of one ledge before the start of the other.

Preferably, the needle holder is a syringe barrel.

Preferably, each ledge is congruent and/or diametrically opposed.

The invention in a second aspect resides broadly in a needle holder as specified above, incorporating a screw-threaded needle or double ended needle.

The invention in a third aspect resides broadly in a needle holder as specified above, incorporating a screw-threaded double ended needle and a blood tube.

### BRIEF DESCRIPTION OF THE DRAWINGS

In order that the invention may be more readily understood and put into practical effect, reference will now be made to the accompanying sheets of drawings wherein:

FIG. 1 is a side view of one embodiment of the present invention showing a syringe barrel incorporating a double ended needle in a bevel up barrel down position;

FIG. 2 is a sectional side view and close up of the embodiment shown in FIG. 1;

FIG. 3 shows various views including a sectional view of the syringe barrel shown in FIGS. 1 and 2 (through line A-A) illustrating a pair of ledges in the bore of the proximal end of the syringe barrel.

FIG. 4 shows a user connecting a double ended needle to the syringe barrel of the embodiment shown in FIGS. 1-3 and subsequently rotating the double ended needle into a bevel up barrel down position by aligning a half closed needle cover associated with the double ended needle with a bevel up indicator on the syringe barrel to achieve a bevel up barrel down position.

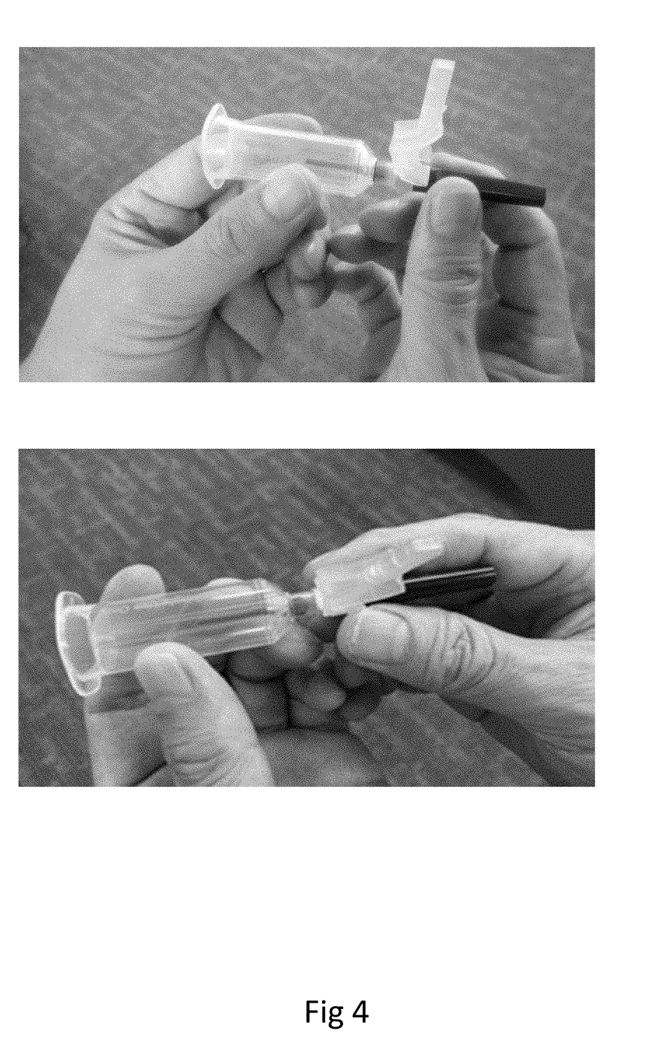

### DESCRIPTION OF THE PREFERRED EMBODIMENT

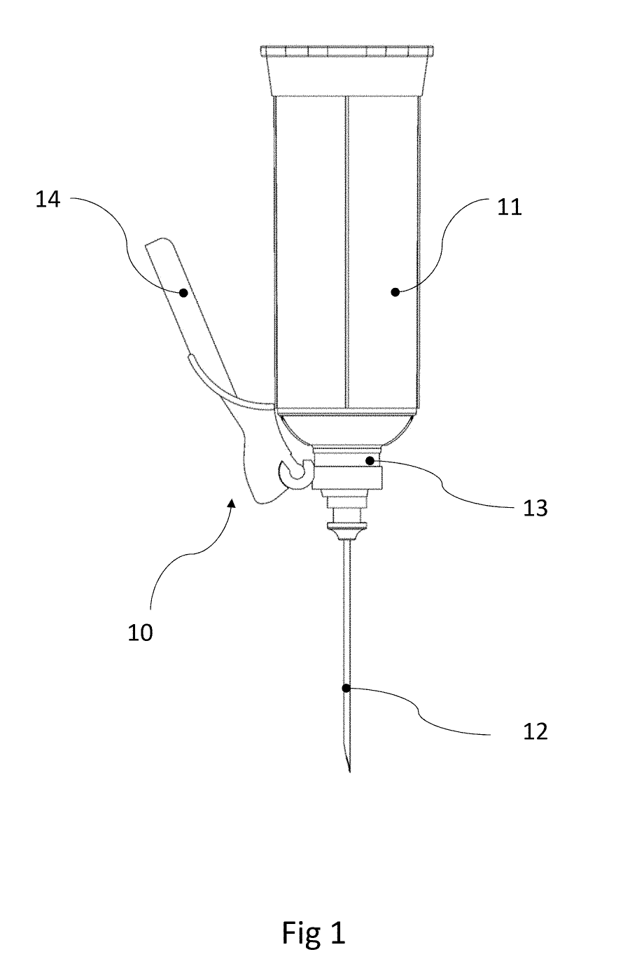

FIG. 1 shows a side view of one embodiment of the present invention which is generally referenced 10. A molded polypropylene needle holder 11 in the form of a modified syringe barrel having a proximal end and a distal end is shown connected to a conventional double ended needle 12 via the male screw-thread (not illustrated in this Fig) of the double ended needle hub 13. Connected to the needle hub 13 is a foldable needle guard 14 in its open position. The exposed end of the double ended needle 12 is in a bevel up position.

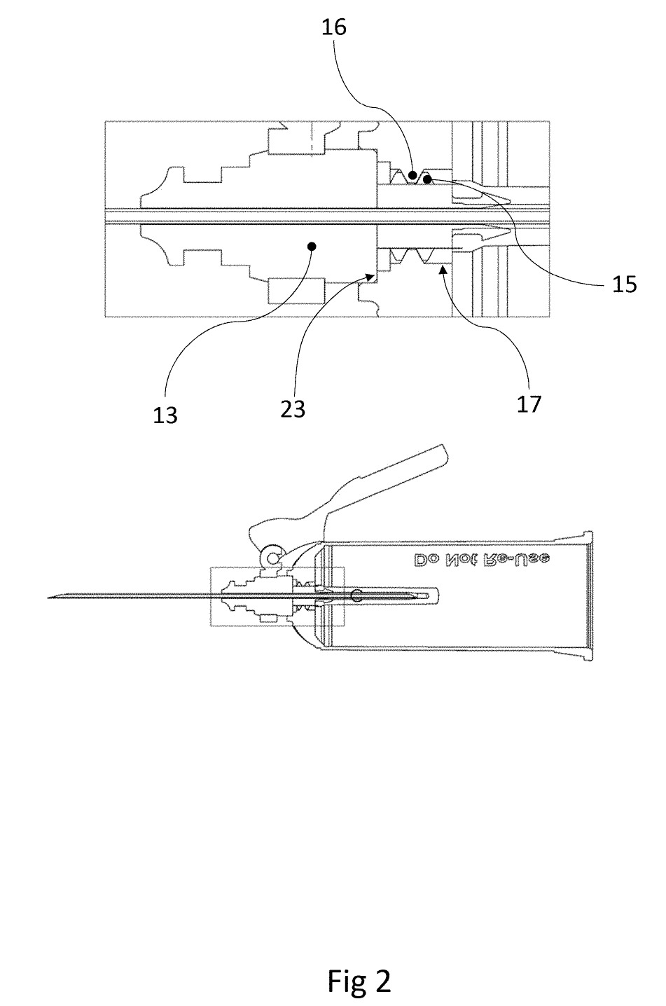

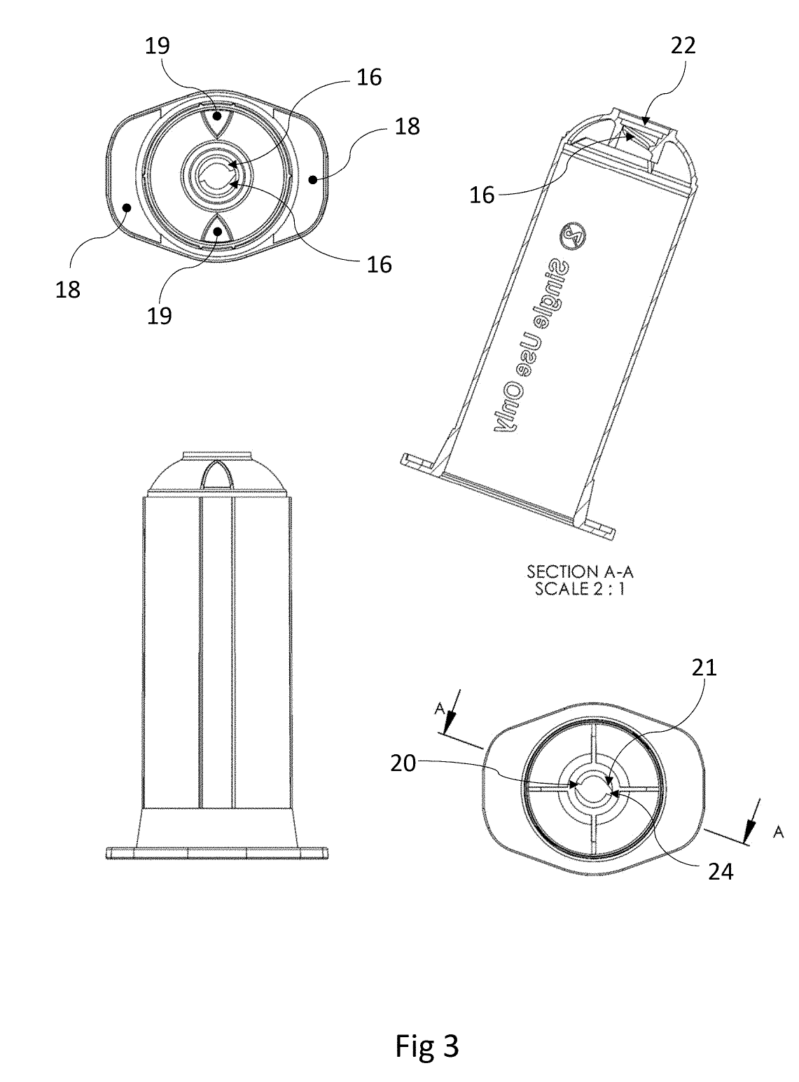

FIG. 2 shows more detail of the male thread 15 of the hub 13 screwed onto a pair of ledges 16 in the bore 17 of the needle holder 11 at its proximal end, the distal end of the needle holder 11 shows a pair of opposed integral finger supports 18 which are shown more clearly in FIG. 3. The ledges 16 are substantially truncated cone shaped in cross-section.

FIG. 3 also shows the exterior proximal end of the needle holder 11 having a pair of molded opposed alignment guides 19 whose function will be described later. As can be seen, the proximal end of the needle holder 11 comprises a circular bore 17 all the way through and concentric with the longitudinal axis of the needle holder 11. The wall of the bore 17 incorporates the pair of integrally molded congruent diametrically opposed ledges 16 in the form of helical ledges.

The path of each respective ledge 16 spans approximately half the circumference of the bore 17, when looking down the longitudinal axis of the needle holder 11, the incline of each ledge being so arranged on the wall as to permit complementary mating contact with the screw-threaded hub 13 of the double ended needle 12. One end of each ledge 16 terminates in substantially a right angled shoulder 20 when viewed down the longitudinal axis of the needle holder 11, whilst the other end when viewed in the same manner terminates in a bevel cut 21. The arrangement between the ledges 16 is that the shoulder 20 of one ledge 16 follows the bevel 21 of the other ledge 16. The proximal end of the needle holder 11 also incorporates a circumferential shoulder 22 to act as a stop member for a complementary shoulder/stop member 23 of the hub 13. Finally, a gap 24 overlies each end of one ledge 20 before the start of the other when viewed down the longitudinal axis of the needle holder 11.

In use, a pre-attached needle for example, is formed by the conventional screw-threaded hub 13 of a double ended needle 12 being screwed to the ledges 16 of the needle holder 11, (the hub 13 threads 15 entering the ledges 16 via the bevel ends 21 of the ledges 16). When this occurs, this may involve the complementary stop members 22 and 23 contacting each other. If the exposed end of the double ended needle is not in a bevel up position at this point, the needle guard 14 is flicked up from its open position to a semi-closed position and on account of a combination of:
a) the gap 24;
b) low modulus of elasticity of the polypropylene of the needle holder 11;
c) dimensions of the ledges 16; and
d) the fact that there are only a pair of substantially semi-circular helical ledges 16 in the bore 17 of the needle holder 11;

a user is able to further rotate either through over turning (or under turning) the hub 13 to align the guard 14 with the apex of double ended needle 12 to a bevel up position using the needle guard 14 on the hub 13 in a half closed position as a guide.

When the semi closed (or upright) guard 14 overlies the apex of either one of the alignment guides 19, the exposed end of the double ended needle 12 will be in a bevel up position and continued rotation of the hub 13 is then stopped.

Furthermore, as the location of the apex of each respective alignment guide 19 is such that it overlies the mid-point between respective finger supports 18, when the exposed end of the double ended needle 12 is in a bevel up position, the needle holder 11 will be in a barrel down position with similar edges of each finger support 18 being adjacent or close to the skin of a patient. At this point, the needle can be pushed into a vein. Once this is done, a blood tube (not illustrated) is pushed into the needle holder 11 incorporating end of the double ended needle 12 and once the septum of the blood tube is pierced, blood will automatically be collected.

1. A needle holder having a proximal end and a distal end, the proximal end being provided with means adapted to secure an integral screw-threaded hub of a needle or a double ended needle, wherein the means comprises a circular wall defining a bore through the proximal end of the needle holder, the wall incorporating a pair of opposed helical ledges, the path of each respective ledge spanning up to half the circumference of the bore, the incline of each ledge being so arranged on the wall as to permit complementary mating contact with a screw-threaded hub of a needle or a double ended needle.

2. The needle holder as claimed in claim 1, wherein one end of each ledge terminates in a right angled shoulder.

3. The needle holder as claimed in claim 1 or claim 2, wherein one end of each ledge terminates in a bevel.

4. The needle holder as claimed in any one of the preceding claims, wherein the needle holder has an alignment guide.

5. The needle holder as claimed in any one of the preceding claims, wherein each ledge has a substantially uniform cross-section throughout its length.

6. The needle holder as claimed in any one of the preceding claims, wherein the needle holder is made of plastics material with a suitable Young's modulus.

7. The needle holder as claimed in any one of the preceding claims, wherein a gap overlies each end of one ledge before the start of the other.

8. The needle holder as claimed in any one of the preceding claims, wherein the needle holder is a syringe barrel.

9. The needle holder as claimed in any one of the preceding claims, wherein one or each ledge is a continuous ledge.

10. The needle holder as claimed in any one of the preceding claims, wherein each ledge is congruent and/or diametrically opposed.

11. A needle holder as claimed in any one of the preceding claims, wherein the needle holder incorporates a screw-threaded needle or double ended needle.

12. A needle holder as claimed in any one of claims 1 to 10, wherein the needle holder incorporates a screw-threaded double ended needle and a blood tube.
